(19) United States
(12) Patent Application Publication
(10) Pub. No.: US 2026/0157937 A1
(43) Pub. Date: Jun. 11, 2026

(54) NATURAL-LOOKING DENTAL PROSTHESIS

(71) Applicant: James R. Glidewell Dental Ceramics, Inc., Newport Beach, CA (US)

(72) Inventors: Sreeram Balasubramanian, Irvine, CA (US); Yan Yang, Wynantskill, NY (US); Shenghan Guan, Irvine, CA (US); Anna Luu, Santa Ana, CA (US); Justin Chi, Irvine, CA (US); Andrew Schissler, Garden Grove, CA (US)

(21) Appl. No.: 19/407,653

(22) Filed: Dec. 3, 2025

Related U.S. Application Data
(60) Provisional application No. 63/727,445, filed on Dec. 3, 2024.

(51) Int. Cl. A61K 6/15; A61C 13/08; A61C 13/083; A61K 6/60; A61K 6/78

(52) U.S. Cl. CPC A61K 6/15; A61C 13/082; A61C 13/083; A61K 6/60; A61K 6/78; A61C 2201/002

(57) ABSTRACT

Methods for making natural-looking dental prosthesis that meet several aesthetic indicators are described, including precoloring methods, compositions, and kits for obtaining a natural-looking dental prosthesis. In one aspect, several different compositions are applied to certain surface areas of a dental prosthesis via a precolor pattern. Exemplary compositions include a colorant composition, an opaque composition, and an incisal composition. The colorant composition may include Zn, Al and at least one coloring agent. The opaque composition may include at least one opaquing agent. The incisal composition may include a Mg component.

### CROSS-REFERENCE TO RELATED APPLICATIONS

This application claims the benefit of and priority to U.S. Provisional Application No. 63/727,445, filed on Dec. 3, 2024. The prior application is incorporated herein by reference in its entirety.

### BACKGROUND

Dental ceramic materials have been widely used for prosthesis because of useful properties, including esthetics, chemical resistance, mechanical stability, and biocompatibility. Good esthetics (i.e., a natural-looking appearance) play a critical role for patient selection of dental prosthesis. Current methods for enhancing the esthetics of a dental prosthesis include porcelain layering and applying multiple stain layers. However, both techniques are overly cumbersome. Precoloring of zirconia can also be used but the current precoloring approaches provide less than desirable esthetics.

### SUMMARY

In one aspect, disclosed herein is a method for coloring a ceramic dental prosthesis, wherein the ceramic dental prosthesis has a total length, comprising:
applying at least one coating of a liquid colorant composition to a portion of an external surface area of the ceramic dental prosthesis, wherein the liquid colorant composition comprises a first opaquing agent comprising Zn, a second opaquing agent comprising Al and at least one coloring agent;
applying at least one coating of an opaque composition to at least a portion of an internal surface of the ceramic dental prosthesis, wherein the opaque composition comprises at least one opaquing agent; and
sintering the coated ceramic dental prosthesis to obtain a fully sintered ceramic body having a density that is at least 98% of the theoretical density,
wherein the coloring of the ceramic dental prosthesis provides:
a drop in L* and C* as defined by a negative slope of L* and C* vs distance from incisal edge to within 1 mm of the incisal edge;
a point of lowest lightness in the 33% of the total length of the ceramic dental prosthesis that is distinguishable with a ΔE of greater than 7 due to both a lower L* and C* when compared with a middle 45-55% of the ceramic dental prosthesis length;
a saturation (S) at a translucent opal halo of the incisal (minimum L*) of no more than 70% that of the middle 45-55% of the ceramic dental prosthesis length; and
an average C* and S throughout the middle third of the ceramic dental prosthesis length that is greater than or equal to C* and S at the border of middle 45-55% of the total length of the ceramic dental prosthesis and 33% of the total length of the ceramic dental prosthesis near the incisal region.

In another aspect, disclosed herein is a coloring system kit for coloring a ceramic dental prosthesis comprising:
(I) at least 4 unique liquid colorant compositions, wherein each colorant composition comprises:
(a) a Zn(NO₃)₂·6H₂O opaquing agent;
(b) an AlCl₃·6H₂O opaquing agent;
(c) an Fe(NO₃)₃·9H₂O coloring agent;
(d) a Ni(NO₃)₂·6H₂O coloring agent;
(e) polypropylene glycol; and
(g) at least one solvent;
(II) at least one unique liquid opaque composition, wherein each opaque composition comprises at least one opaquing agent; and
(III) a liquid incisal solution comprising a Mg component.

In a further aspect, disclosed herein is a method comprising:
testing a ceramic dental prosthesis, wherein the ceramic dental prosthesis has a total length, for color criteria:
a drop in L* and C* as defined by a negative slope of L* and C* vs distance from incisal edge to within 1 mm of the incisal edge;
a point of lowest lightness in the 33% of the total length of the ceramic dental prosthesis that is distinguishable with a ΔE of greater than 7 due to both a lower L* and C* when compared with a middle 45-55% of the ceramic dental prosthesis length;
a saturation at a translucent opal halo of the incisal (minimum L*) of no more than 70% that of the middle 45-55% of the ceramic dental prosthesis length;
an average C* and S throughout the middle third of the ceramic dental prosthesis length that is greater than or equal to C* and S at the border of middle 45-55% of the total length of the ceramic dental prosthesis and 33% of the total length of the ceramic dental prosthesis near the incisal region;
an opacity greater than or equal to 65%, calculated as the ratio of the minimum Y luminance in the middle 40-80% of the ceramic dental prosthesis length (relative to the incisal edge) when a black stump is fit in the intaglio surface to that of the same ceramic dental prosthesis measured without a stump;
and providing a ceramic dental prosthesis that satisfies the color criteria to an end-user.

The foregoing will become more apparent from the following detailed description, which proceeds with reference to the accompanying figures.

### BRIEF DESCRIPTION OF THE DRAWINGS

FIG. 1 is an overview table describing five indicators for a natural-looking dental ceramic body.

FIG. 2 illustrates an image captured by the SpectroShade Micro II imaging spectrophotometer (MHT Medical High Technologies), highlighting the opalescent and opaque halo regions of a natural-looking dental ceramic body.

FIG. 3 is a data table demonstrating the presence of, or lack of presence, of a first indicator of a natural-looking dental ceramic body. In the table, “e” or “Ex” designate inventive examples; “ce” or “CE” designate a comparative example.

FIG. 4 is a data table demonstrating the presence of, or lack of presence, of a second indicator of a natural-looking dental ceramic body. In the table, “e” or “Ex” designate inventive examples; “ce” or “CE” designate a comparative example.

FIG. 5 is a data table demonstrating the presence of, or lack of presence, of a third indicator of a natural-looking dental ceramic body. In the table, “e” or “Ex” designate inventive examples; “ce” or “CE” designate comparative example.

FIG. 6 is a data table demonstrating the presence of, or lack of presence, of a fourth indicator of a natural-looking dental ceramic body. In the table, “e” or “Ex” designate inventive examples; “ce” or “CE” designate comparative example.

FIG. 7 is a data table demonstrating the presence of, or lack of presence, of a fifth indicator of a natural-looking dental ceramic body. In the table, “e” or “Ex” designate inventive examples; “ce” or “CE” designate comparative examples.

FIGS. 8A-8D shows an example of an inventive method for applying several different compositions to several different surface areas of an anterior dental prosthesis (e.g., a crown).

FIG. 9 shows an example of applying an opaque liquid to the intaglio labial surface of an anterior dental prosthesis (e.g., a crown).

FIG. 10 depicts an anterior dental prosthesis having facial surface locations identified as 1, 2, 3, 4, and 5.

FIG. 11A is a table of several inventive examples and of several comparative examples alongside FIG. 11B providing colorant composition details of the shaded zirconia blocks.

FIG. 12 is a graph showing an example of a sintering process used for inventive examples.

FIG. 13 is a table showing examples of body coloring compositions used in inventive examples and comparative examples as disclosed herein.

FIG. 14 is a table showing examples of opaque compositions used in inventive examples and comparative examples as disclosed herein.

FIG. 15 is a table showing an example of an incisal composition used in inventive examples and comparative examples as disclosed herein.

FIGS. 16A-16C are graphs showing all examples (FIG. 16A-16B) that meet aspects of Chroma value measured in a method as disclosed herein, and comparative examples (FIG. 16C).

FIGS. 17A-17C are graphs showing all examples (FIG. 17A-17B) that meet aspects of Saturation value measured in a method as disclosed herein, and comparative examples (FIG. 17C).

FIGS. 18A-18C are graphs showing all examples (FIG. 18A-18B) that meet aspects of ΔE measured and calculated in a method as disclosed herein, and comparative examples (FIG. 18C).

FIGS. 19A-19C are graphs showing all examples (FIG. 19A-19B) that meet aspects of L* measured in a method as disclosed herein, and comparative examples (FIG. 19C).

FIGS. 20A-20C are graphs showing aspects of a* measured in a method as disclosed herein for all examples (FIG. 20A-20B) and comparative examples (FIG. 20C).

FIGS. 21A-21C are graphs showing aspects of b* measured in a method as disclosed herein, for all examples (FIG. 21A-21B) and comparative examples (FIG. 21C).

FIG. 22 shows the Al and Zn compound content and opacity as defined herein for select examples and comparative examples.

### DETAILED DESCRIPTION

In general, a natural-looking dental prosthesis (e.g., a crown) has a translucent incisal area, visible lobes, and a smooth transition from the incisal area to the body of the dental prosthesis. Disclosed herein are methods and liquid compositions for applying to ceramic dental prosthesis prior to sintering.

Unless indicated otherwise, as used herein, “body” is defined as starting at 67% length of the dental prosthesis to the gingival area.

Unless indicated otherwise, as used herein, “precoloring” refers to coloring of a dental prosthesis prior to final sintering of the dental prosthesis.

Disclosed herein are precoloring methods, compositions, and kits for obtaining a natural-looking dental prosthesis. In one aspect, several different compositions are applied to certain surface areas of a dental prosthesis via a precolor pattern. The compositions include a colorant composition, an opaque composition, and an incisal composition. The colorant composition includes Zn, Al and at least one coloring agent. The opaque composition includes at least one opaquing agent. The incisal composition includes a Mg component. In certain embodiments, application of the incisal composition is optional.

The dental prosthesis may be in the form of a crown, veneer, single or multi-unit bridge, implant-supported partial or full-arch denture, and the like, that attach to the support structure of a patient, such as an implant abutment or natural tooth preparation. In certain embodiments, the dental prosthesis is a crown. In certain embodiments, the dental prosthesis is a crown for an anterior tooth. In certain embodiments, the dental prosthesis is a crown for a maxillary central incisor, a mandibular central incisor, a lateral incisor, or a cuspid.

In certain embodiments, the dental restoration after precoloring as disclosed herein has a transmittance greater than 60%, at 700 nm when measured on a 1 mm thick sintered body at the incisal region. In certain embodiments, the precoloring disclosed herein is applied to a yttria-stabilized zirconia dental ceramic having greater than 5 mol % yttria.

Also disclosed herein is a pre-color kit that includes at least one dental restoration block (e.g., a crown), colorant composition(s), opaque composition(s), and incisal composition(s). The kit may further include a least one composition applicator (e.g., a marker or felt-tip pen that can be loaded with a liquid composition).

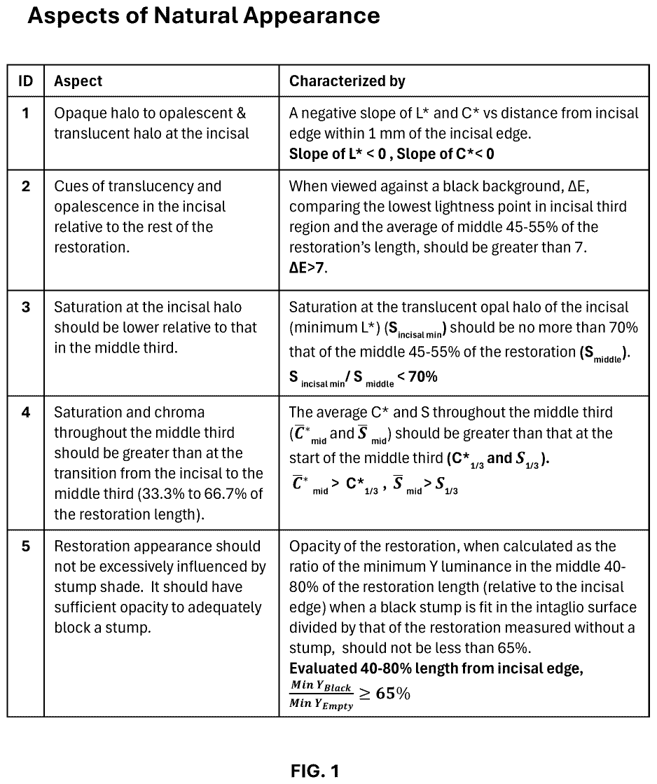

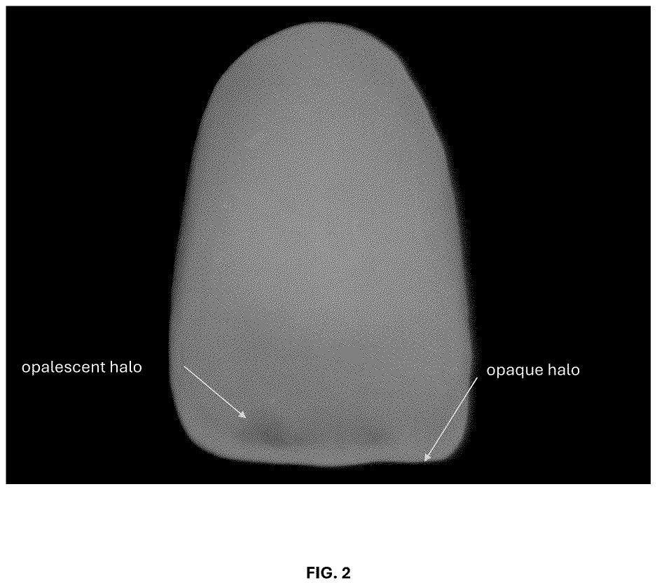

It has been discovered that a natural-looking dental prosthesis should meet several indicators. FIG. 1 summarizes several indicators defining a natural-looking dental prosthesis. FIG. 2 is an image captured by the SpectroShade Micro II showing opalescent halo and opaque halo region on a natural-looking dental ceramic body.

CIELAB color space is used to define the indicators of a natural-looking dental ceramic body. In the CIELAB color space:
L* represents lightness with the range of 0-100;
a* represents redness-greenness of color where positive a* is redness and negative a* is greenness; and
b* represents yellowness-blueness of the color where the positive b* is yellowness and negative b* is blueness.

### Color Difference ΔE is Calculated as Follows:

$$Δ E = {( Δ {L}^{* 2} + Δ {a}^{* 2} + Δ {b}^{* 2} )}^{1 / 2}$$

wherein,
ΔL*—lightness difference
Δb*—yellowness or blueness difference, and
Δa*—redness or greenness difference
   Chroma is defined as the colorfulness of an area judged in proportion of the brightness of a similarly illuminated area that appears grey, white, or highly transmitting (a white point). In the CIELAB color space, chroma refers to the color intensity of a color. It represents how far the color is from the neutral gray axis, where colors have no chroma.

### Chroma is Calculated as Follows:

$${C}^{*} = \sqrt{{a}^{* 2} + {b}^{* 2}}$$

Saturation is the colorfulness of an area judged in proportion to its brightness. Using CIELAB color metrics, Saturation may be approximated as the ratio of chroma to lightness, expressing how vivid the color appears relative to its brightness.

### Saturations is Calculated as Follows:

$$S = \frac{{C}^{*}}{{L}^{*}}$$

CIELAB color metrics for examples and comparative examples described herein were obtained from spectral image data of the labial faces of glazed crowns collected using a SpectroShade Micro II imaging spectrophotometer. Prior to collecting spectral image data, the SpectroShade Micro II was calibrated in accordance with built-in calibration instructions provided with the instrument—using the white and green tiles on the docking base provided with the unit. Crowns were imaged over a dark background (the AC/DC switching adaptor supplied with the SpectroShade Micro II; MEAN WELL ENTERPRISES, GS40A15-P1M). A small dot of wax was used to support the crown by the cingulum upon the dark background such that the labial face was approximately level with the dark background surface and exposed for spectral imaging. The SpectroShade Micro II (with mouthpiece attached) was then aligned by hand and used to capture a spectral image measurement file for each crown.

Each prosthesis was scanned as-is (empty) and then again with a black stump clay (Foam Clay—Black; CALPALMY) stuffed into the intaglio pocket and pressed flush against the labial intaglio wall.

SpectroShade measurement files were subsequently uploaded to PC and analyzed using the SpectroShade Analysis software. CIELAB color space values were collected for each scan via a sweep across the middle of the restoration face. Starting from approximately 0.25 mm from the incisal edge, each sweep collected CIELAB metric averages of areas approximately 1 mm wide and in contiguous 0.25 mm long increments and ending within 0.60 mm of the gingival edge.

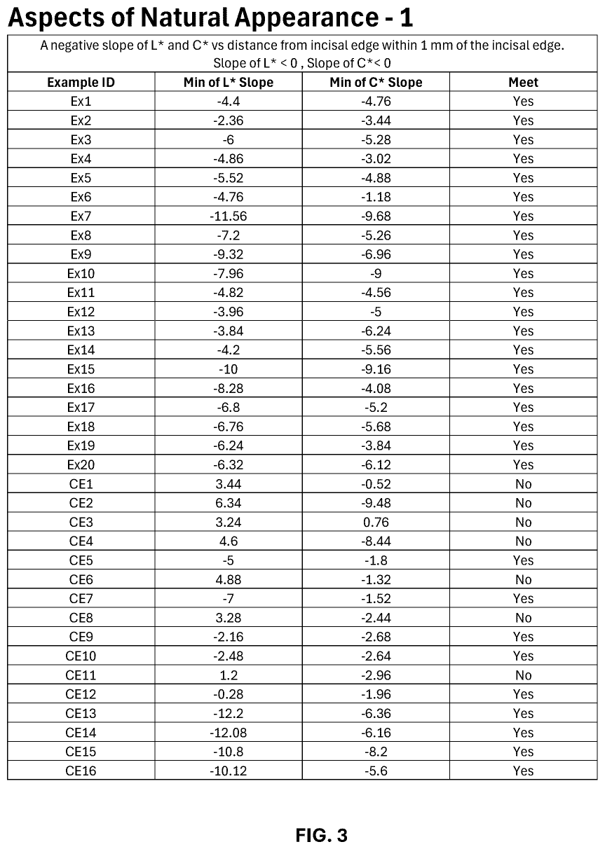

In one indicator, the incisal region of a prosthesis should exhibit an opaque halo to opalescent and translucent halo appearance. As shown in FIG. 3, the presence of such a halo is characterized by a drop in L* and C* as defined by a negative slope of L*, \dot{L}* and C, Ċ* vs distance from incisal edge to within 1 mm of the incisal edge. \dot{L}* and Ċ* are calculated as follows:

$$\dot{{L}^{*}} = \frac{( {L}_{1}^{*} - {L}_{0}^{*} )}{( {l}_{1} - {l}_{0} )}$$

$$\dot{{C}^{*}} = \frac{( \sqrt{{a}_{1}^{* 2} + {b}_{1}^{* 2}} - \sqrt{{a}_{0}^{* 2} + {b}_{0}^{* 2}} )}{( {l}_{1} - {l}_{0} )}$$

L₀* is the L* value from the first measurement point
L₁* is the lowest L* value measured within 1 mm of the incisal edge
a*₀, b*₀ are the corresponding a* value and b* value at L₀*
a*1, b*₁ are the corresponding a* value and b* value at L₁*
l₀ is the distance from incisal at the first measurement point
l₁ is the distance from incisal at the lowest L* value measured within 1 mm of the incisal edge

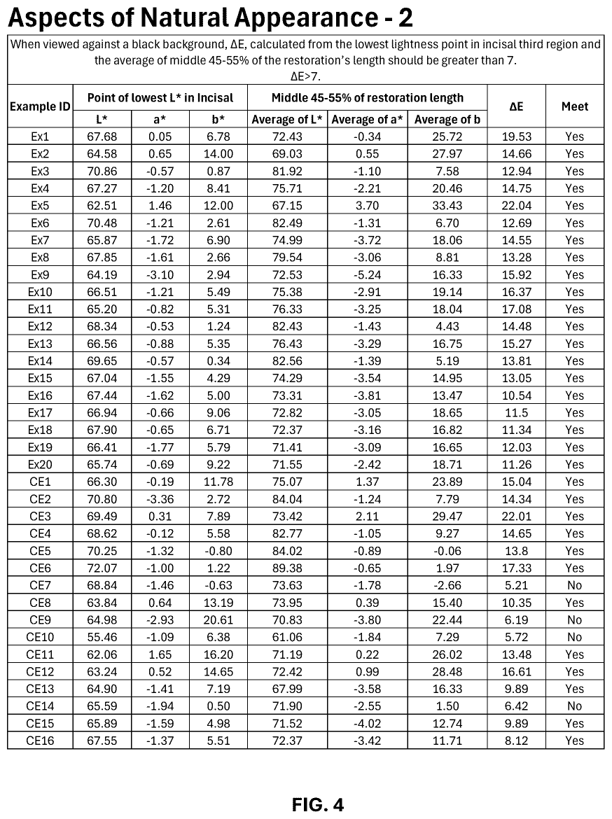

Another indicator is cues of translucency and opalescence in the incisal relative to the other areas of the dental prosthesis. When viewed against a black background, the incisal 3^{rd} should have a region of lowest lightness. As shown in FIG. 4, when compared with the middle 45-55% of the restoration's length, the point of lowest lightness in the incisal 3^{rd} should be distinguishable with a ΔE of greater than 7 due to both a lower L* and C*. The “incisal 3^{rd}” ΔE is defined as 33% of the total length of the dental prosthesis near the incisal region. AE, calculated as follows, should be greater than 7:

$$Δ E = \sqrt{\begin{matrix}{( {L}_{avg 45 - 55 \%}^{*} - {L}_{min L Incisal}^{*} )}^{2} + {( {a}_{avg 45 - 55 \%}^{*} - {a}_{min L Incisal}^{*} )}^{2} + \\ {( {b}_{avg 45 - 55 \%}^{*} - {b}_{min L Incisal}^{*} )}^{2}\end{matrix}}$$

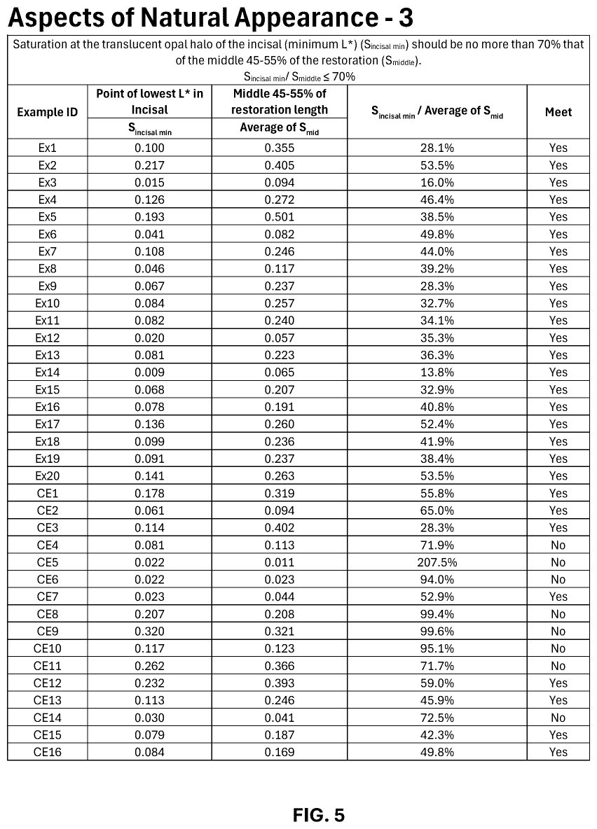

A further indicator is that the natural incisal halo should be lower in saturation relative to the middle third. The natural incisal halo should be less saturated than the body. As shown in FIG. 5, the presence of this indicator is characterized by having a saturation at the translucent opal halo of the incisal (minimum L*) of no more than 70% that of the middle 45-55% of the prosthesis.

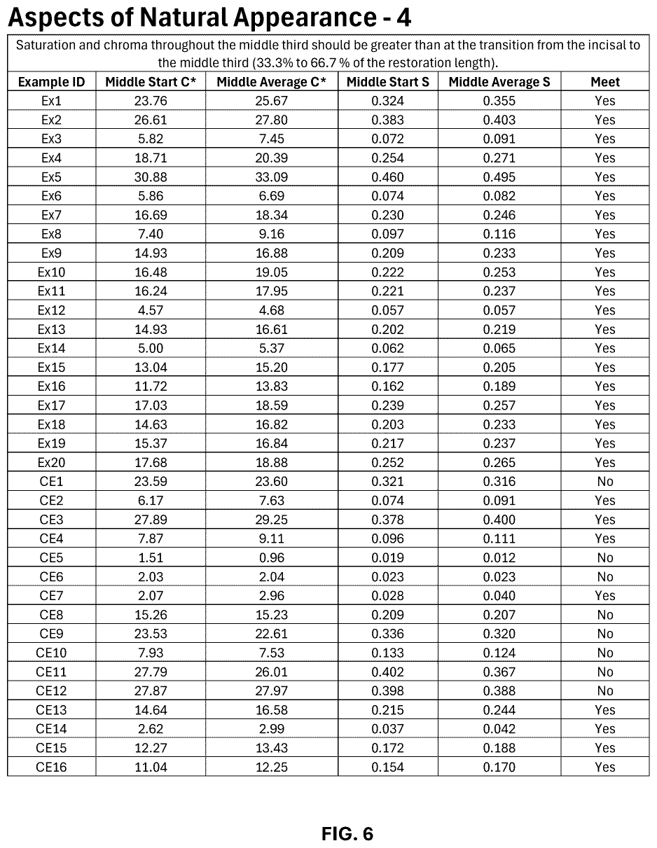

An additional indicator is that saturation and Chroma throughout the middle third of a prosthesis should be greater than at the transition from the incisal to the middle third. Saturation and chroma should peak near the end of the incisal third. As shown in FIG. 6, the presence of this indicator is characterized by having an average C* and S throughout the middle third that is greater than or equal to C* and S at the start of the middle third (border of middle third and incisal third).

Another indicator is that the blocking power or opacity of the prosthesis be sufficient to block a stump from showing through. Opacity, or the blocking power of an object, may be evaluated as a ratio of the Y luminance factors taken from reflectance measurements of the object against contrasting backgrounds. Typically, this comprises measurement of the object against a black, non-reflective background and another measurement of the object against either a white, diffusely-reflective background or a background of greater thickness of the object material. Opacity is then taken as the ratio of the luminance factor taken against the non-reflective background to that of the typically more-reflective one. The closer the result is to 100%, the greater the opacity of the object.

In order to approximate opacity in this way, the Y tristimulus luminance may be determined from reflectance measurements of the object. The SpectroShade Micro II outputs values for selected areas in CIELAB color space based on standard illuminant D65 and the 1931 2-degree standard observer color functions to obtain the representative perceptual color stimulus metrics. Knowing this, we may convert output L* measurements back into Y luminance factor from the following equations:

$$f ( Y / {Y}_{n} ) = \frac{{L}^{*} + 16}{116}$$

$$( Y / {Y}_{n} ) = {f ( Y / {Y}_{n} )}^{3} if f ( Y / {Y}_{n} ) > \frac{24}{116}$$

$$( Y / {Y}_{n} ) = ( f ( Y / {Y}_{n} ) - \frac{16}{116} ) ( \frac{108}{841} ) if f ( Y / {Y}_{n} ) \leq \frac{24}{116}$$

Where Y is the tristimulus luminance of the test object color stimulus and Yₙ is the tristimulus luminance of a white object color stimulus under the same illumination and observation conditions. For standard illuminant D65 and based on the 1931 2-degree standard observer color functions, Yₙ=100.

For each crown, L* measurements were taken incrementally across the crown face from incisal to gingival, Y luminance was calculated from the L* data, and the minimum Y value within the 40-80% region relative to the incisal edge was collected from a scan taken with nothing in the intaglio (Y_{empty}) and another was collected from a scan taken with a black stump clay (Foam Clay—Black; CALPALMY) stuffed into the intaglio pocket and pressed flush against the labial intaglio wall (Y_{black}). From these two luminance factors from measurements taken for the ceramic dental prosthesis, opacity is calculated as follows:

$$Opacity = \frac{{Y}_{black}}{{Y}_{empty}}$$

For an esthetic ceramic dental prosthesis, opacity should be no less than 65% and preferably no less than 70% in order to adequately block underlying stumps. FIG. 7 indicates the luminance factors, resulting opacity ratio, and whether the example or counter example meets aspect 5 of natural appearance.

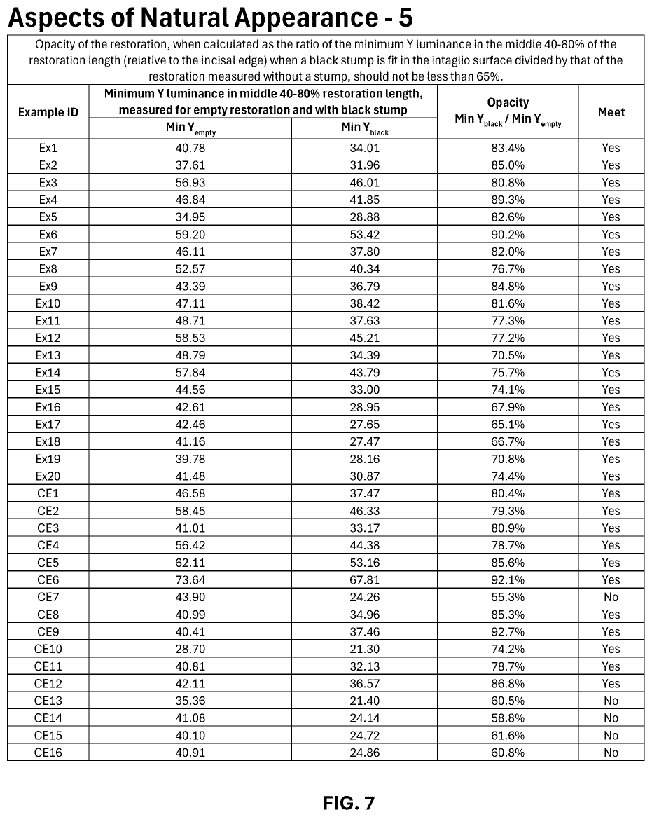

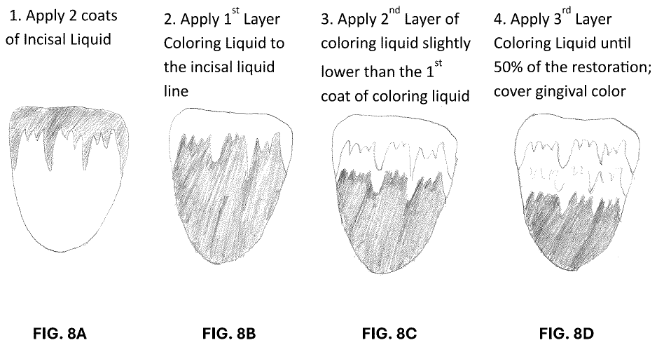

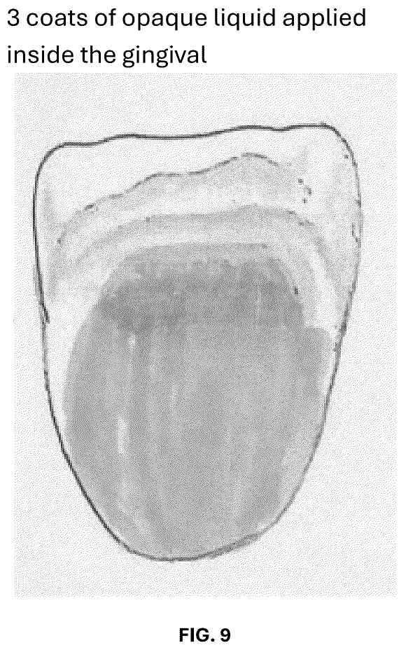

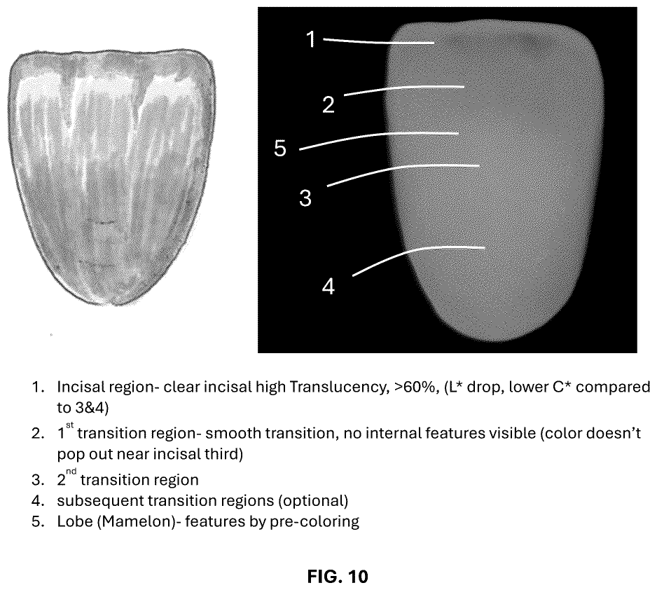

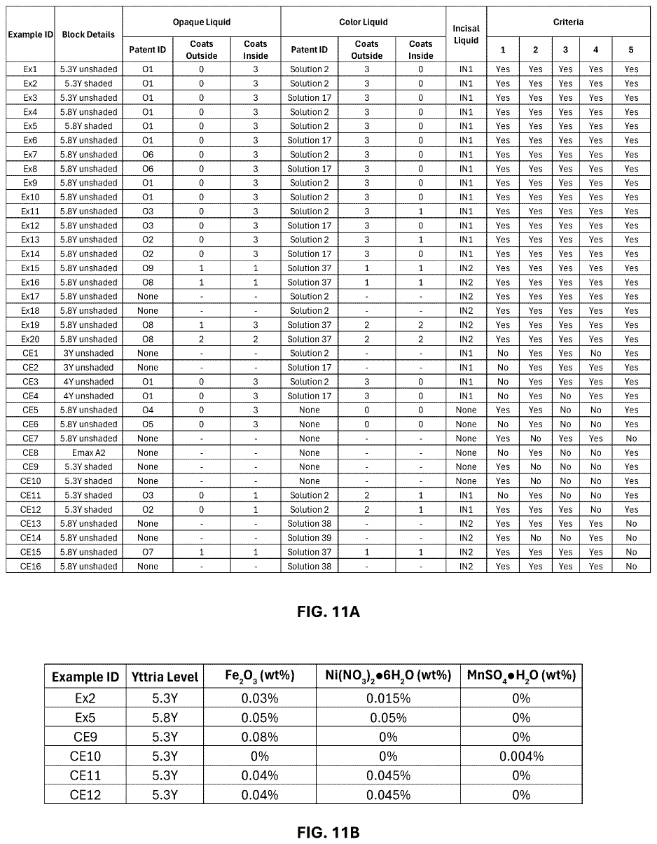

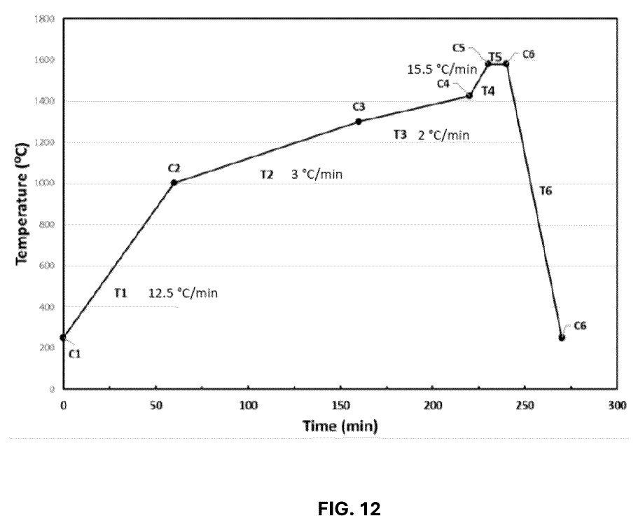

The methods disclosed herein involve applying a colorant composition and an opaque composition, and optionally an incisal composition, to dental prosthesis surfaces. The colorant composition and the opaque composition can be applied in any order. In certain embodiments, the methods involve sequentially applying an incisal composition, a colorant composition, and an opaque composition to dental prosthesis surfaces. FIGS. 8A-8D and 9 show an example of an inventive method for applying several different compositions to several different surface areas of a dental prosthesis (e.g., a crown).

As shown in the example of FIGS. 8A-8D, an incisal composition is applied initially to coat the incisal area of a dental prosthesis (see FIG. 8A). A colorant composition is subsequently applied to coat the body area of the dental prosthesis from the gingival edge to the edge line of the previously coated incisal area. More than one coating of the colorant composition can be applied. For instance, two, three or four coatings can be applied. The same colorant composition, or different colorant compositions, can be used for each coating. For example, as shown in FIG. 8B, a first layer of the colorant composition is applied to coat the body of the dental prosthesis from the gingival edge to the edge line of the previously coated incisal area. As shown in FIG. 8C, a second layer of the colorant composition is applied to coat the body of the dental prosthesis, but at an upper line slightly lower than the upper line of the first layer of colorant composition. As shown in FIG. 8D, a third layer of the colorant composition is applied to coat 40-70% of the body surface, starting from the gingival edge. In certain embodiments, the incisal composition and the colorant composition are applied in a pattern to provide a lobes effect.

As shown in FIG. 9, an opaque composition is applied to the inside of the dental prosthesis to coat the gingival area. In certain embodiments more than one coating of the opaque composition can be applied. For example, two, three or four coatings can be applied. The same opaque composition, or different opaque compositions, can be used for each coating.

The incisal composition includes a Mg component. The Mg component may be in the form of a hydroxide, alkoxide, oxide, or a salt of magnesium. Illustrative magnesium salts include nitrate, sulfate, carbonate, chloride and hydrates thereof. In some embodiments, the magnesium component is Mg(NO₃)₂·(H₂O)ₓ, wherein x is 0, 2 or 6. In some embodiments, the component may be dissolved in an organic solvent or a non-organic solvent (e.g., water).

In some embodiments, the incisal composition may also include HNO₃.

In some embodiments, the incisal composition may also include a wetting agent, such as n-propanol, glycerol, ethylene glycol, polyethylene glycol (e.g., PEG 200 or PEG 400), or polypropylene glycol (e.g., PPG 400) may be added to incisal composition to control the penetration depth of the Mg component in the porous ceramic body. In some embodiments, the incisal composition may also include at least one food coloring additive. In some embodiments, the incisal composition may include a yttrium salt.

In some embodiments, the incisal composition may include 5 wt % to 70 wt %, more particularly 10 wt % to 60 wt %, and most particularly 10 wt % to 30 wt %, of the Mg component, based on the total weight of the incisal composition. The incisal composition may include 0.1 wt % to 5 wt %, more particularly 0.1 wt % to 3 wt %, and most particularly 0.5 wt % to 2 wt %, of the wetting agent, or a mixture of wetting agents, based on the total weight of the incisal composition.

In some embodiments, the magnesium-containing dopant converts into an oxide form such as MgO and may be incorporated into the zirconia lattice upon sintering.

Examples of incisal compositions are further described in US Patent Application Publication No. 2023/0202931, which is incorporated herein by reference in its entirety.

In certain embodiments, the colorant composition includes at least one coloring agent and at least one opaquing agent. In certain embodiments, the colorant composition is a liquid solution that includes at least one coloring agent, at least one opaquing agent, at least one wetting agent, and at least one solvent.

The coloring agent(s) can produce dentally acceptable shade effects on dental materials, even with high translucency. For example, the coloring agent(s) can achieve a final color in the sintered yttria-stabilized zirconia ceramic material that matches a shade from a VITA A1-D4® Classical Shades shade guide or VITA 3D Master Shade Guide, such as 0M1, 0M2 or 0M3 bleach shades, (available through VITA North America) when measured according to the shade match evaluation test method provided herein.

The coloring agent can be a transition metal-containing material, such as a metal complex or metallic compound, for example, a metallic salt. Illustrative transition metal-containing materials suitable for use as coloring agents include oxides or salts of one or more elements selected from Fe, Ni, Cu, Mn, Co, Cr, and Mo, or a combination thereof. Metal-containing materials may comprise an anion such as acetate, oxalate, sulfate, carbonate, halide (e.g., chloride or fluoride), nitrate, phosphate or citrate. In certain embodiments, the coloring agent is a hydrate of the metallic salt.

The coloring agent can be a rare earth metal-containing material, such as a rare earth metal complex or rare earth metallic compound, for example, a rare earth metallic salt. Illustrative rare earth metal-containing materials suitable for use as coloring agents include oxides or salts of one or more elements selected from Pr, Nd, Er, Ce, and Tb, or a combination thereof. Rare earth element-containing materials may include an anion such as acetate, oxalate, sulfate, carbonate, halide (e.g., chloride or fluoride), nitrate, phosphate or citrate.

In one embodiment, a coloring agent in the form of a metallic salt is selected that is soluble in an aqueous liquid colorant composition. The coloring agent may be added to the colorant composition in the form of a solid or a liquid.

Illustrative coloring agents include Fe(NO₃)₃·9H₂O, Ni(NO₃)₂·6H₂O, CuCl₂·2H₂O, MnSO₄·H₂O, Er(NO₃)₃·6H₂O, CoCl₂·6H₂O, Nd(NO₃)₃·6H₂O, Cr(NO₃)₃·9H₂O, and Tb(NO₃)₃·6H₂O.

The colorant composition may include 0.03 wt % to 70 wt %, more particularly 2 wt % to 60 wt %, more particularly 5 wt % to 30 wt %, most particularly 5 wt % to 12 wt % of the coloring agent, or a mixture of coloring agents, based on the total weight of the colorant composition. In certain embodiments, the colorant composition includes 0.3 wt % to 25 wt %, more particularly 2 wt % to 13 wt %, and most particularly 5 wt % to 10 wt %, of an Fe-containing coloring agent (e.g., Fe(NO₃)₃·9H₂O). In certain embodiments, the colorant composition includes 0.3 wt % to 2.5 wt % and most particularly 0.3 wt % to 2 wt % of a Ni-containing coloring agent (e.g., Ni(NO₃)₂·6H₂O). In certain embodiments, the colorant composition includes 0.01 wt % to 1 wt %, and more particularly 0.05 wt % to 0.5 wt %, of a Cu-containing coloring agent (e.g., CuCl₂·2H₂O). In certain embodiments, the colorant composition includes 0 to 0.5 wt %, more particularly 0.03 wt % to 0.2 wt %, of a Mn-containing coloring agent (e.g., MnSO₄·H₂O).

The colorant composition may include 0.05 g/L to 500 g/L, more particularly 0.1 g/L to 250 g/L, and most particularly 20 g/L to 80 g/L, or 150 g/L to 210 g/L, of the metal ion of the coloring agent, or a mixture of coloring agents.

The opaquing agent(s) can modify the lightness of dental materials and create opaque effects on the body part of the dental restorations, producing more esthetic effects to better mimic the nature tooth appearance. The opaquing agent can be a material containing Zn, Al, Si, or a combination thereof. The opaquing agent may be a complex or a compound, for example, a salt or an oxide. The opaquing agent may include an anion such as acetate, oxalate, sulfate, carbonate, halide (e.g., chloride or fluoride), nitrate, phosphate or citrate. In certain embodiments, the opaquing agent is a Zn-containing material or an Al-containing material. In certain embodiments, the colorant composition includes both a Zn-containing material and an Al-containing material.

In one embodiment, an opaquing agent in the form of a salt is selected that is soluble in an aqueous liquid colorant composition. The opaquing agent may be added to the colorant composition in the form of a solid or a liquid.

Illustrative opaquing agents include Zn(NO₃)₂·6H₂O, AlCl₃·6H₂O, and tetraethyl orthosilicate (TEOS).

The colorant composition may include 1 wt % to 30 wt %, more particularly 10 wt % to 15 wt %, or 25 wt % to 30 wt %, of the opaquing agent, or a mixture of opaquing agents, based on the total weight of the colorant composition. In certain embodiments, the colorant composition includes 0 wt % to 30 wt %, more particularly 5 wt % to 11 wt %, and most particularly 6 wt % to 8 wt % of a Zn-containing opaquing agent (e.g., Zn(NO₃)₂·6H₂O). In certain embodiments, the colorant composition includes 0.1 wt % to 10 wt %, more particularly 1 wt % to 10 wt %, more particularly 2 wt % to 5 wt %, and most particularly 2 wt % to 3.5 wt %, of an Al-containing opaquing agent (e.g., AlCl₃·6H₂O). In certain embodiments, the weight ratio of Zn(NO₃)₂·6H₂O/AlCl₃.6H₂O is in the range of 1 to 10, more particularly in the range of 2 to 3 or 6.5 to 7.5. In certain embodiments, the weight ratio of metal ion Zn/Al is in the range of 3 to 15, or more particularly in the range of 3.5 to 5.5 or 13 to 14.

The colorant composition may include 0 g/L to 300 g/L, more particularly 10 g/L to 200 g/L, and even more particularly 20 g/L to 150 g/L, and most particularly 25 g/L to 80 g/L, of the opaquing agent, or a mixture of opaquing agents.

The colorant composition may include 250 to 2800 mmol/L, more particularly 250 to 600 mmol/L, or 400 to 1200 mmol/L, or 400 to 2800 mmol/L of Zn ion. The colorant composition may include 100 to 2000 mmol/L and more particularly 100 to 300 mmol/L, or 250 to 2000 mmol/L of Al ion.

A wetting agent, such as n-propanol, glycerol, ethylene glycol, polyethylene glycol (e.g., PEG 200 or PEG 400), or polypropylene glycol (e.g., PPG 400) may be added to the liquid colorant composition to control the penetration depth of the coloring agent and/or the opaquing agent in the porous ceramic body.

The colorant composition may include 0.1 wt % to 5 wt %, more particularly 0.1 wt % to 3 wt %, and most particularly 0.5 wt % to 2 wt %, of the wetting agent, or a mixture of wetting agents, based on the total weight of the colorant composition.

The solvent can be aqueous or non-aqueous. Illustrative solvents include water, organic solvents such as ethanol alcohol, isopropyl alcohol, acetone, and mixtures thereof.

In certain embodiments, the colorant composition includes:
(a) at least one opaquing agent comprising a material comprising Zn, Al, Si, or a combination thereof,
(b) at least one coloring agent comprising a material comprising Fe, Ni, Cu, Mn, Co, Cr, Mo, Pr, Nd, Er, Ce, Tb, or a combination thereof;
(c) at least one wetting agent; and
(d) at least one solvent.

In certain embodiments, the colorant composition includes:
(a) 5 wt % to 10 wt % of a first opaquing agent comprising Zn, based on the total weight of the composition;
(b) 0.5 wt % to 5 wt % of a second opaquing agent comprising Al, based on the total weight of the composition;
(c) 3 wt % to 15 wt % of a first coloring agent comprising Fe, based on the total weight of the composition;
(d) 0.1 wt % to 4 wt % of a second coloring agent comprising Ni, based on the total weight of the composition;
(e) at least one wetting agent; and
(f) at least one solvent. In certain embodiments, the solvent is present in an amount of 20 to 90 wt %, based on the total weight of the composition.

In certain embodiments, the colorant composition includes:
(a) a Zn(NO₃)₂·6H₂O opaquing agent;
(b) an AlCl₃·6H₂O opaquing agent;
(c) an Fe(NO₃)₃·9H₂O coloring agent;
(d) a Ni(NO₃)₂·6H₂O coloring agent;
(e) polypropylene glycol; and
(g) at least one solvent.

The colorant compositions are homogenous solutions. The colorant compositions also are stable to ensure final shade consistency. In particular, the colorant compositions do not exhibit precipitation over a commercially useful time period (for example, up to 5 years, more particularly up to 3 years, and most particularly up to 2 years).

Examples of colorant compositions are further described in US Patent Application Publication No. 2023/0404860, which is incorporated herein by reference in its entirety.

The opaque composition is applied to an internal, or fitting, surface of a dental prosthesis that has been shaped for attachment to the support structure. The opaque composition masks the visibility of a dental support structure, underlying a highly translucent dental prosthesis

A liquid opaque composition comprises a solvent and one or more opaquing agents that can penetrate the porosity of a porous ceramic body in an amount effective for reducing the visibility an underlying support structure through the sintered form of the porous dental prosthesis to which the opaque composition is applied. The liquid opaque composition may be, for example, a solution, suspension or colloid.

A opaquing agent comprises a metal-containing component, such as a metal complex or metallic compound, for example, a metallic salt. Metal-containing components suitable for use as opaquing agents comprise oxides or salts of one or more elements selected from Zn, Ti, La, Al, Ca, Mn, Mg, Si, Sc and Sr, or a combination thereof. Metal-containing components may comprise an anion such as acetate, oxalate, sulfate, carbonate, chloride, nitrate, phosphate or citrate.

Examples of opaquing agents include, but are not limited to, aluminum chloride (e.g., AlCl₃.6H₂O), zinc sulfate (e.g., ZnSO₄·7H₂O), zinc nitrate (e.g., Zn(NO₃)₂·6H₂O), scandium chloride (e.g., ScCl₃·6H₂O), magnesium nitrate (e.g., Mg(NO₃)₂·6H₂O), lanthanum nitrate (e.g., La(NO₃)₃·6H₂O), titanium butoxide (Ti[O(CH₂)₃CH₃]₄), potassium silicate (e.g., K₂SiO₃), tetrasodium pyrophosphate (e.g., Na₄P₂O₇·10H₂O), calcium nitrate (e.g., Ca(NO₃)₂·4H₂O, and strontium nitrate (e.g., Sr(NO₃)₂). The opaquing agents may be used alone, or two or more may be combined to form an opaque composition.

The liquid component of the opaque composition may be aqueous or non-aqueous, including but not limited to water, inorganic solvents, such as water, organic solvents such as ethanol alcohol, isopropyl alcohol, and combinations thereof. The liquid opaque composition may comprise from 0.4 wt % to 50 wt % of the opaquing agent, based on the total weight of the liquid opaque composition, or from 5 wt % to 40 wt %, or from 8 wt % to 30 wt %, or from 10 wt % to 30 wt %, or from 20 wt % to 30 wt %, of the opaquing agent based on the total weight of the liquid opaque composition. In one embodiment, a liquid opaque composition comprises from 0.4 wt % to 50 wt % of AlCl₃.6H₂O, based on the total weight of the liquid opaque composition. In one embodiment, an aqueous liquid opaque composition comprises a combination of aluminum chloride and zinc nitrate. For example, an aqueous opaque composition comprises from 0.4 wt % to 40 wt % of AlCl₃.6H₂O and from 5 wt % to 40 wt % of Zn(NO₃)₂·6H₂O, based on the total weight of the liquid opaque composition.

The liquid opaque composition may comprise from 5 g/L to 500 g/L of the metal ion of the opaquing agent, or from 5 g/L to 450 g/L, or from 5 g/L to 400 g/L, or from 5 g/L to 300 g/L, of the metal ion of the opaquing agent in the liquid opaque composition. In one embodiment, an aqueous opaque composition comprises from 5 g/L to 300 g/L of aluminum. In a further embodiment, an aqueous opaque composition comprises from 5 g/L to 300 g/L of aluminum and from 10 g/L to 200 g/L of zinc.

The liquid opaque composition may comprise 100 to 2100 mmol/L and more particularly 600 to 2000 mmol/L, or 250 to 2000 mmol/L of Al ion. The liquid opaque composition may comprise 200 to 3400 mmol/L and more particularly 400 to 2800 mmol/L of Zn ion.

A wetting agent, such as n-propanol, glycerol, ethylene glycol or polyethylene glycol (e.g., PEG 200 or PEG 400) may be added to the opaque composition in an amount of approximately 0.1 wt % to 5 wt %, based on the total weight of the opaque composition, to control the penetration depth of the opaquing agent in the porous ceramic body.

A dental crown comprises an internal space or cavity that has been shaped to fit over a patient's support structure, such as a natural tooth, tooth preparation or implant abutment. The liquid opaque composition may be applied to an intaglio surface of the crown that fits over the support structure. The liquid opaque composition may be applied to the internal bottom surface that may be adjacent the incisal region of the patient's tooth preparation upon installation.

Examples of opaque compositions are further described in U.S. Pat. No. 12,005,131, which is incorporated herein by reference in its entirety.

Also disclosed herein is a coloring system kit for coloring a dental prosthesis that includes:
(I) at least 4 unique liquid colorant compositions, wherein each colorant composition comprises:
(a) a Zn(NO₃)₂·6H₂O opaquing agent;
(b) an AlCl₃·6H₂O opaquing agent;
(c) an Fe(NO₃)₃·9H₂O coloring agent;
(d) a Ni(NO₃)₂·6H₂O coloring agent;
(e) polypropylene glycol; and
(g) at least one solvent;
(II) at least one unique liquid opaque composition, wherein each opaque composition comprises at least one opaquing agent; and
(III) a liquid incisal solution comprising a Mg component.

In certain embodiments, the coloring system kit includes 4 to 8 unique liquid colorant compositions.

In certain embodiments, the coloring system kit includes at least 16 unique liquid colorant compositions.

The compositions can produce dentally acceptable shade effects on dental materials, even materials with high translucency. Highly translucent, sintered yttria-stabilized zirconia dental ceramics may include materials having between 50% and 75% transmittance, or between 58% and 67% transmittance, or 60% and 65% transmittance, at 700 nm when measured on a 1 mm thick sintered body.

A dental block can be used for producing a dental prosthesis. The dental block includes an yttria-stabilized zirconia green body having a density between 52% to 62% theoretical density and having an amount of yttrium oxide between 5 mol % and 8.5 mol %. In an embodiment, a zirconia green body has a median particle size of less than 350 nm, for example, where D₍₅₀₎ is from 150 nm to 350 nm, or 200 nm to 350 nm, such as, wherein D₍₅₀₎ is from 220 nm to 320 nm, or wherein D₍₅₀₎ is from 250 nm to 300 nm.

Methods are provided for heating zirconia ceramic green bodies in a pre-sintering process to form zirconia ceramic bisqued bodies having high densities and low porosities that are suitable for subtractive manufacturing processes such as machining, milling, and the like, that are suitable for use in forming dental restoration devices. Porous bisques bodies may be sintered by novel sintering methods, providing sintered bodies with enhanced physical properties.

In one embodiment, a dental block for producing a dental prosthesis comprises a zirconia bisqued body having a relative density between 55% and 65% of theoretical density. In some embodiments, the median pore size of bisque bodies is less than 200 nm, or less than 150 nm, less than 100 nm, such as between 40 nm and 80 nm, or between 45 nm and 75 nm, when measured according to the methods described herein.

In some embodiments, fully sintered ceramic bodies made from these ceramic powders having from 5.4 mol % yttria to 7.0 mol % yttria, have greater than 62 percent transmittance at 700 nm (when measured on a 1 mm thick sintered ceramic body). In other embodiments, fully sintered ceramic bodies made from these ceramic powders having between 5.5 mol % yttria and 6.9 mol % yttria, have greater than 65 percent transmittance at 700 nm (measured on a 1 mm thick fully sintered ceramic body). In further embodiments, sintered ceramic bodies made from these powders having between 5.7 mol % yttria and 6.3 mol % yttria, have greater than 68 percent transmittance at 700 nm, (when measured on a sintered 1 mm thick body by the methods described herein). In a further embodiment, the sintered yttria-stabilized zirconia ceramic materials have a flexural strength greater than or equal to, 300 MPa, or greater than or equal to, 500 MPa. In a further embodiment, fully sintered bodies having a flexural strength from 300 MPa to 750 MPa, may comprise an average grain size greater than or equal to 1 μm, such as from 1 μm to 30 μm, or from 1 μm to 15 μm, or greater than or equal to 8 μm, such as from 8 μm to 20 μm, when measured by the methods provided herein.

An unshaded zirconia sintered body is provided that comprises sintered yttria-stabilized zirconia ceramic material that has a total light transmittance value of at least 59% at 700 nm (for a 1 mm thick fully sintered ceramic body), such as between 59% and 78%, or between 59% and 75%, or between 59% and 73%, or between 59% and 71%, and a flexural strength greater than 500 MPa, that was made from yttria-stabilized zirconia ceramic material comprising at least 5 mol % yttria, or at least 5.2 mol % yttria, or at least 5.3 mol % yttria, or at least 5.4 mol % yttria, such as between 5 mol % yttria and 8 mol % yttria. or between 5.2 mol % yttria and 7.8 mol % yttria, or 5.4 mol % yttria and 7.5 mol % yttria.

In some embodiments, sintered yttria-stabilized zirconia ceramic materials may comprise at least 5.2 mol % yttria, and have a total light transmittance value of at least 60% at 700 nm (when a sintered 1 mm thick body of the ceramic material is measured at 700 nm) have flexural strength values greater than 300 MPa, or greater than 500 MPa, such as between 300 MPa and 750 MPa, or between 300 MPa and 600 MPa, or between 500 MPa and 750 MPa, or at least 600 MPa, or at least 625 MPa, or at least 650 MPa, or at least 700 MPa, or between 600 MPa and 750 MPa. The sintered yttria-stabilized zirconia ceramic materials may further comprise at least 5.3 mol % yttria or at least 5.5 mol % yttria, such as between 5 mol % yttria and 7.5 mol % yttria, or between 5.3 mol % yttria and 6.0 mol % yttria, or between 5.5 mol % yttria and 7.0 mol % yttria, or between 5.5 mol % yttria and 7.5 mol % yttria.

In some embodiments, sintered yttria-stabilized zirconia ceramic material having a total light transmittance at 700 nm (for a 1 mm thick fully sintered ceramic body) of greater than 62%, such as between 62% and 75%, such as between 62% and 73%, or between 62% and 71%, or between 62% and 69%, or between 63% and 75%, or between 64% and 75%, or between 64% and 73%, or between 64% and 71%, or between 65% and 75%, or between 65% and 73%, or between 68% and 75%. In these embodiments, the sintered yttria-stabilized zirconia ceramic materials may have flexural strength values greater than 500 MPa, such as between 500 MPa and 750 MPa, or at least 600 MPa, such as between 600 MPa and 750 MPa. In these embodiments, the sintered yttria-stabilized zirconia ceramic material may comprise at least 5 mol % yttria, such as at least 5.4 mol % yttria, or at least 5.5 mol % yttria, for example, between 5.5 mol % yttria and 6.0 mol % yttria, or between 5.5 mol % yttria and 7.0 mol % yttria, or between 5.5 mol % yttria and 7.5 mol % yttria.

In some embodiments, a zirconia ceramic body that has a total light transmittance value between 60% and 69%, or between 60% and 67%, or greater than 62%, such as between 62% and 69%, or between 62% and 67%, at 700 nm (for a 1 mm thick sample), may comprise an yttria-stabilized zirconia ceramic material having at least 6.5 mol % yttria, such as between 6.5 mol % yttria and 7.0 mol % yttria, or between 6.5 mol % yttria and 6.9 mol % yttria, and may have a flexural strength greater than 500 MPa.

In another embodiment, an unshaded zirconia sintered body that comprises sintered yttria-stabilized zirconia ceramic material having a total light transmittance value of at least 59% at 700 nm (for a 1 mm thick sample), a flexural strength greater than 500 MPa, made from yttria-stabilized zirconia ceramic material comprising at least 5.2 mol % yttria, such as between 5.4 mol % yttria and 7.5 mol % yttria, has a fracture toughness between 1.6 and 3.0 MPa·m^{1/2}.

Further examples and description of high translucency ceramic zirconia materials are disclosed in U.S. Pat. No. 11,161,789, which is incorporated herein by reference in its entirety.

The compositions may be applied by techniques such as painting by brushing, or by dipping, or dripping, compositions onto the porous dental prosthesis. Compositions may be applied by known techniques for distributing liquid compositions onto ceramic surfaces, including coating with a marker or felt-tip pen that is loaded with the liquid mixture, or by use of a sponge.

Prior to the application of the compositions, bisque stage dental prostheses may be unshaded, having the color of natural zirconia materials, which may appear unnaturally white upon sintering if no colorant or staining is applied. Alternatively, shaded bisque stage dental prostheses may be obtained that are made from shaded ceramic powder that provides a dentally acceptable shade upon sintering. The compositions applied to at least one surface of a shaded dental prosthesis may alter the final color or shade.

Coloring agents also may be added directly to the ceramic powder to create shaded sintered zirconia ceramic materials having dentally acceptable shades after sintering to theoretical density. As used herein, unshaded zirconia ceramic materials refer to materials in which no coloring agent has been added, and unshaded zirconia ceramic materials often have a bright white appearance conventionally considered esthetically unsuitable for use as a dental restoration without the addition of further coloring or staining. Shaded zirconia ceramic materials comprise additives that may include, but are not limited to metal-containing oxides, salts, or other compounds or complexes that include erbium (Er), terbium (Tb), chromium (Cr), cobalt (Co), iron (Fe), manganese (Mn), nickel (Ni), praseodymium (Pr), copper (Cu), and/or other coloring metal ions in an amount to obtain desired dental shades in final sintered restorations. In some embodiments, shaded yttria-stabilized zirconia ceramic material that has been sintered to approximately full theoretical density match a shade tab from a VITA Classical A1-D4® shade guide or VITA Bleached Shades shade guide, such as 0M1, 0M2 or 0M3 bleach shades, (available through VITA North America) when measured according to the shade match evaluation test method provided herein. In some embodiments, coloring compositions may be applied to the ceramic body after formation of the green, bisque or sintered ceramic body, and may comprise a coloring agent having at least one metal included, but not limited to, Tb, Er, Cr, Fe, Mn, Ni, Pr, Cu or Co, and combinations thereof. In some embodiments, the amount of coloring agent in a sintered ceramic body may be from 100 ppm to about 2000 ppm, or 200 ppm to 1500 ppm, (measured as metal ion) per gram of the yttria-stabilized zirconia ceramic material.

In some embodiments, the composition(s) applied to the outer surface and/or internal side surfaces of a prosthesis may penetrate below the surface for a distance of at least 10000 μm, or at least 5000 μm, or at least 3000 μm, or between 0.01 μm and 3000 μm, or between 0.1 μm and 2000 μm, increasing the concentration of coloring agent(s) and/or opaquing agent(s) in this region.

Penetration of the coloring agent(s) and/or opaquing agent(s) into the ceramic prosthesis may be detected by energy dispersive spectroscopy (EDS) analysis of a cross-section of a dental prosthesis. Points along a line from the facial surface to the internal surface may be analyzed for the concentration of the metal element of the agent. In one embodiment, the concentration of metal attributable to the agent may be between 0.001 wt % and 10 wt %, or between 0.004 wt % and 6 wt %, or between 0.004 wt % and 3 wt % and 4.5 wt % to 6 wt % when analyzed by EDS according to methods described herein. In one embodiment, the concentration of element attributable to the agent may be between 0.01 wt % and 5 wt %, or between 1 wt % and 3 wt %, or between 1 wt % and 1.5 wt %, and between 2.5 wt % and 3 wt %, when analyzed by EDS according to methods described herein.

Dental prostheses may comprise zirconia ceramic materials stabilized by 2 mol % to 10 mol % yttria. Yttria-stabilized zirconia ceramic material may be stabilized, for example, from 3 mol % yttria to 7.5 mol % yttria, from 4.0 mol % yttria to 7.5 mol % yttria, from 4 mol % yttria to 7 mol % yttria, from 5 mol % yttria to 8 mol % yttria, from 5 mol % yttria to 7.5 mol % yttria, from 5 mol % yttria to 7 mol % yttria.

In certain embodiments, sintered zirconia ceramic materials may be stabilized by 3 mol % to 8 mol % yttria. Starting materials for wet forming processes may include, but are not limited to, ceramic powder, dispersant, and deionized water to form ceramic slurries. Yttria-stabilized zirconia ceramic material in the slurry may comprise up to 7.5 mol % yttria, or up to 8.5 mol % yttria, for example, from 5 mol % yttria to 8.5 mol % yttria, from 5 mol % yttria to 8 mol % yttria, from 5 mol % yttria to 7.5 mol % yttria, 5 mol % yttria to 6.4 mol % yttria, from 5 mol % yttria to 5.6 mol % yttria, from 5.1 mol % yttria to 6.4 mol % yttria, from 5.2 mol % yttria to 7.5 mol % yttria, from 5.2 mol % yttria to 7.0 mol % yttria, from 5.4 mol % yttria to 7.5 mol % yttria, from 5.4 mol % yttria to 7.0 mol % yttria, from 5.5 mol % yttria to 7.5 mol % yttria, from 5.5 mol % yttria to 7 mol % yttria, from 5.5 mol % yttria to 6.9 mol % yttria, from 5.5 mol % yttria to 6 mol % yttria, from 5.5 mol % yttria to 5.9 mol % yttria, from 5.6 mol % yttria to 6.3 mol % yttria, from 5.7 mol % yttria to 6.3 mol % yttria, from 5.8 mol % yttria to 6.3 mol % yttria, from 6 mol % yttria to 8.5 mol % yttria, from 6 mol % yttria to 8 mol % yttria, from 6.0 mol % yttria to 7.5 mol % yttria, from 6 mol % yttria to 7 mol % yttria, from 6.0 mol % yttria to 6.8 mol % yttria, from 6.0 mol % yttria to 6.3 mol % yttria, from 6.2 mol % yttria to 7.5 mol % yttria, from 6.4 mol % yttria to 7.5 mol % yttria, from 7 mol % yttria to 8.5 mol % yttria, or from 7.2 mol % to 8.4 mol % yttria, to stabilize zirconia.

Zirconia ceramic material may comprise a mixture of unstabilized zirconia and stabilized zirconia ceramic materials. The term stabilized zirconia ceramic herein includes fully stabilized and partially stabilized zirconia. Specific examples include zirconia with no yttria, or yttria-stabilized zirconia including, but not limited to, commercially available yttria-stabilized zirconia, for example, from Tosoh USA, such as Tosoh TZ-8YS and Tosoh TZ-PX430. The calculated amount of yttria (e.g., yttria mol %) in zirconia ceramic material may vary from ‘nominal’ values implied by commercial nomenclature (e.g. 8YS). The mol % yttria in zirconia ceramic material may be calculated, for example, based on compositional information received from manufacturer certification.

Dental prosthetic shapes may be formed as green bodies or bisqued state bodies. Green body manufacturing methods may include dry forming processes, such as uniaxial pressing and cold isostatic pressing, and wet forming processes, including but not limited to, pressure-casting, slip-casting, filter pressing, and centrifugal casting methods. A green body manufacturing method such as a slip-casting process, may include the process steps of selecting starting materials; mixing and comminuting the starting materials to form a slurry; and casting the slurry to form a desired green body form, such as the shape of a milling blocks. Methods for making zirconia dental prosthesis materials suitable for use herein may be found in commonly owned patents and patent publications, including U.S. Pat. Nos. 9,434,651, 9,790,129, and U.S. Pat. Pub. 2018/0235847, the subject matter of each is hereby incorporated by reference in its entirety.

Yttria-stabilized zirconia ceramic materials used as starting materials to form millable blocks may, optionally, include a small amount of alumina (aluminum oxide, Al₂O₃) as an additive. For example, some commercially available yttria-stabilized zirconia ceramic material includes alumina at concentrations of from 0 wt % to 2 wt %, or from 0 wt % to 0.25 wt %, such as 0.1 wt %, relative to the zirconia material. Other optional additives of the ceramic starting material may include coloring agents to obtain shaded zirconia ceramic powder that may be formed by, for example, casting or pressing into shaded ceramic blocks that have a dentally acceptable shade or pre-shade upon sintering.

Dispersants used to form ceramic suspensions or ceramic slurries to form green bodies by slip-casting manufacturing methods such as those described herein, function by promoting the dispersion and/or stability of the slurry and/or decreasing the viscosity of the slurry.

Dispersion and deagglomeration may occur through electrostatic, electrosteric, or steric stabilization. Examples of suitable dispersants include nitric acid, hydrochloric acid, citric acid, diammonium citrate, triammonium citrate, polycitrate, polyethyleneimine, polyacrylic acid, polymethacrylic acid, polymethacrylate, polyethylene glycols, polyvinyl alcohol, polyvinyl pyrillidone, carbonic acid, and various polymers and salts thereof. These materials may be either purchased commercially or prepared by known techniques. Specific examples of commercially available dispersants include Darvan® 821-A ammonium polyacrylate dispersing agent commercially available from Vanderbilt Minerals, LLC; Dolapix™ CE 64 organic dispersing agent and Dolapix™ PC 75 synthetic polyelectrolyte dispersing agent commercially available from Zschimmer & Schwarz GmbH; and Duramax™ D 3005 ceramic dispersant commercially available from Dow Chemical Company.

Zirconia ceramic and dispersant starting materials added to deionized water may be mixed to obtain a slurry. Slurries may be subjected to a comminution process for mixing, deagglomerating and/or reducing particle size of zirconia ceramic powder particles. Comminution may be performed using one or more milling process, such as attritor milling, horizontal bead milling, ultrasonic milling, or other milling or comminution process, such as high shear mixing or ultra-high shear mixing capable of reducing zirconia ceramic powder particle sizes described herein.

In one embodiment, a zirconia ceramic slurry may undergo comminution by a horizontal bead milling process. Media may comprise zirconia-based beads, for example, having a diameter of 0.4 mm. A suspension or slurry having a zirconia ceramic solids loading of about 60 wt % to about 80 wt % and a dispersant concentration from 0.002 gram dispersant/gram zirconia ceramic powder to 0.01 gram dispersant/gram zirconia ceramic powder, may be used to prepare the zirconia ceramic slurry. Milling processes may include, for example, a flow rate of 1 kg to 10 kg zirconia ceramic powder/hour, such as, approximately 6 kg zirconia ceramic powder/hour where, for example, approximately 6 kg of zirconia ceramic material is milled for approximately one hour, at a mill speed of approximately 1500 rpm to 3500 rpm, for example, approximately 2000 rpm.

In some embodiments, where commercially available zirconia ceramic is used as starting materials to prepare the ceramic slurry, the measured median particle size, or particle size distribution at D₍₅₀₎ may be about 200 nm to 600 nm, or greater than 600 nm, which includes agglomerations of particles of crystallites having a crystallite size of about 20 nm to 40 nm. As used herein, the term “measured particle size” refers to measurements obtained by a Brookhaven Instruments Corp. X-ray disk centrifuge analyzer. By processes described herein, an initial particle size distribution at, for example, a D₍₅₀₎ of about 200 nm to 600 nm, or greater than 600 nm, may be reduced to provide a zirconia ceramic material contained in a slurry having a median particle size where D₍₅₀₎ is from 100 nm to 600 nm, such as, wherein D₍₅₀₎ is from 150 nm to 350 nm, or from 220 nm to 320 nm or wherein D₍₅₀₎ is from 250 nm to 300 nm. In some embodiments, after comminution processes ceramic slurries comprise particle size distributions wherein D₍₁₀₎ is from 100 nm to 250 nm, or D₍₁₀₎ is from 120 nm to 220 nm, or D₍₁₀₎ is from 120 nm to 200 nm, and D₍₉₀₎ of zirconia particles is less than 800 nm, or D₍₉₀₎ is in the range of 250 nm to 425 nm.

By processes described herein, zirconia ceramic material may comprise an initial median particle size, for example, a D₍₅₀₎ of less than 400 nm, which upon comminution may provide a slurry comprising a zirconia ceramic material having a median particle size where D₍₅₀₎ is from 100 nm to 350 nm, such as, wherein D₍₅₀₎ is from 80 nm to 280 nm. Yttria-stabilized zirconia ceramic material comprising mixtures of two or more yttria stabilized zirconia ceramic materials each having different initial median particle sizes, may be comminuted as a mixture in a slurry by the processes described herein. Reduced particle sizes and/or narrow ranges of comminuted zirconia ceramic material, in combination with the dispersants describe above, may provide cast parts with a higher density and smaller pores that form sintered bodies having higher translucency and/or strength than those obtained by way of conventional pressing and slip-casting processes.

Zirconia ceramic slurries may be cast into a desired shape, such as a solid block, disk, near net shape, or other shape. Ceramic slurries may be poured into a porous mold (e.g., plaster of Paris or other porous/filtration media) having the desired shape, and cast, for example, under the force of capillary action, vacuum, pressure, or a combination thereof (for example, by methods provided in US 2013/0313738, which is hereby incorporated by reference in its entirety). Green bodies may form a desired shape as water contained in the slurry is absorbed/filtered through the porous media. Excess slurry material, if any remaining, may be poured off the green body. Green bodies removed from molds may dry, for example, at room temperature in a controlled, low humidity environment. Dental milling blanks may be cast, for example, as a solid block, disk or near-net-shape, having dimensions suitable for use in milling or grinding single unit or multi-unit restorations, such as crowns, veneers, bridges, partial or full-arch dentures, and the like.

Manufacturing processes described herein may provide green bodies having relative densities ρ_{R} greater than 48%, such as from 52% to 65% relative density, or such as from 56% to 62% relative density. As used herein, the term “relative density” (ρ_{R}) refers to the ratio of the measured density ρ_{M} of a sample (g/cm³) to the theoretical density ρ_{T} (3 YSZ—6.083 g/mL; 5 YSZ—6.037 g/mL; 7 YSZ—5.991 g/mL).

Green bodies may be partially consolidated to obtain bisqued bodies by a heating step. Bisquing methods include heating or firing green bodies, such as green bodies in the shape of blocks to obtain, for example, porous bisqued blocks. In some embodiments, relative densities of bisque blocks do not increase more than 5% over the green body density. In some embodiments, the ceramic bodies are bisque heated so that the difference between the relative densities of the bisque body and the green body is 3% or less. Resulting bisqued bodies may be fully dried and have strength sufficient to withstand packaging, shipping, and milling, and in some embodiments, have a hardness value of less than or equal to 0.9 GPa, when tested by the hardness test method described herein. Bisque firing steps may include heating the green body at an oven temperature of from 800° C. to 1100° C. for a holding period of about 0.25 hours to 3 hours, or about 0.25 hours to 24 hours, or by other known bisquing techniques. In some embodiments, bisque processes comprise heating green bodies in an oven heated at an oven temperature of 900° C. to 1000° C. for 30 minutes to 5 hours.

Processes described herein may provide a bisqued body having a relative density ρ_{R} greater than or equal to 48%, such as from 48% to 62%, or from 54% to 60%. Bisqued bodies may have a porosity of less than or equal to 45%, such as from 35% to 45%, or from 38% to 42%, or from 38% to 41%. As used herein, the term “porosity”, expressed as percent porosity above, is calculated as: percent porosity=1−percent relative density. A dental block for producing a dental prosthesis includes a zirconia bisqued body having a density of between 56% to 65% of theoretical density and having a porosity of between 35% and 44%, such as between 38% and 41%.

In some embodiments, the median pore size of bisque bodies is less than 200 nm, or less than 150 nm, less than 100 nm, such as from 30 nm to 150 nm, or from 30 nm to 80 nm, or from 35 nm to 40 nm, or from 40 nm to 80 nm, or from 40 nm to 70 nm, or from 45 nm to 75 nm, or from 45 nm to 50 nm, or from 50 nm to 80 nm, or from 50 nm to 75 nm, or from 55 nm to 80 nm, or from 55 nm to 75 nm, when measured according to the methods described herein. As used herein, the term “median pore diameter” refers to the pore diameter measurements obtained from a bisqued body via mercury intrusion performed with an Autopore V porosimeter from Micromeritics Instrument Corp.

Conventional subtractive processes, such as milling or machining processes known to those skilled in the art, may be used to shape a bisqued zirconia ceramic body or milling block into a pre-sintered dental restoration. For dental applications, a pre-sintered restoration may include a dental restoration such as a crown, a multi-unit bridge, an inlay or onlay, a veneer, a full or partial denture, or other dental restoration. For example, bisque stage blocks milled to form bisque-stage dental restorations having anatomical facial surface features including an incisal edge or biting surface, anatomical dental grooves and cusps, and are sintered to densify the bisque-stage restoration into the final dental restoration that may permanently installed in the mouth of a patient. In alternative embodiments, bisque-stage zirconia ceramic bodies are shaped into near-net-shape blocks having generic sizes and shapes that are sintered to theoretical density prior to machining into a final patient-specific dental restoration. The sintered near-net-shape bodies may be prepared having a shape and/or size that is suitable for range of similarly sized and shaped final restoration products.

Dental prostheses may be shaped from porous, pre-sintered blocks by conventional subtractive processes, such as milling or machining processes known to those skilled in the art. The blocks may be shaped in a crown, a multi-unit bridge, an inlay or onlay, a veneer, a full or partial denture, or other dental prosthesis.

After treating bisque stage dental prostheses by applying the compositions as disclosed herein, the bisque stage bodies may be “fully sintered” under atmospheric pressure to a density that is at least 98% of the theoretical density of a sintered body. Sintering may occur at oven temperatures in the range of 1200° C. to 1900° C., or 1400° C. to 1600° C., or 1400° C. to 1450° C. Hold times (dwell times) at a temperature within a sintering temperature range may be from 1 minute to 48 hours, such as from 10 minutes to 5 hours, or from 30 minutes to 4 hours, or from 1 hour to 4 hours, or from 1 hour to 3 hours, or from 2 hours to 2.5 hours. Other sintering processes include multi-step sintering processes described in commonly owned U.S. Pat. Pub. 2019/0127284, filed Oct. 31, 2018, hereby incorporated herein by reference in its entirety. Multi-step sintering processes may comprise one or more temperature gradients within a sintering temperature range, with each gradient having the same or different ramp rates, reaching oven temperatures at or above 1200° C., such as from 1200° C. to 1900° C. Multi-step sintering methods may optionally having no hold time within a sintering temperature range, or one hold time or multiple hold times at or above 1200° C. Multi-step sintering processes may have multiple temperature peaks at or above 1200° C., and at least one temperature steps that is between 25° C. to 600° C. lower, or between 50° C. to 400° C. lower, than a preceding or subsequent temperature peak. Hold times at temperature peaks may be between 0 minutes and 30 minutes, and a lower temperature step between two temperature peaks may have a hold time between 2 minutes and 5 hours.

### EXAMPLES

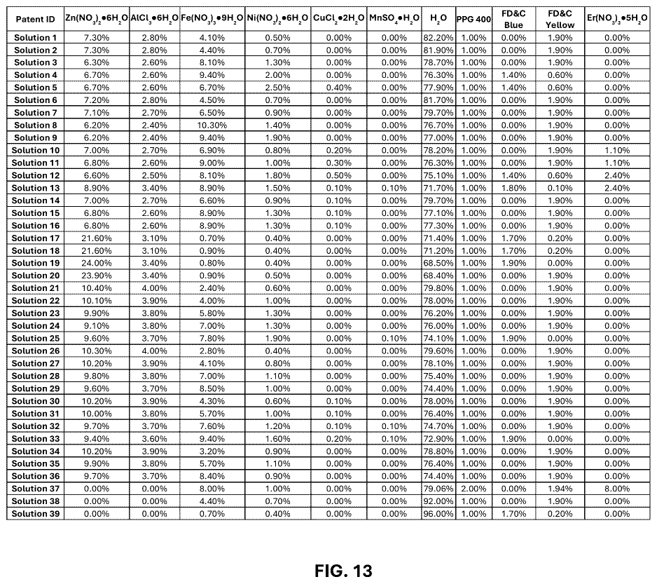

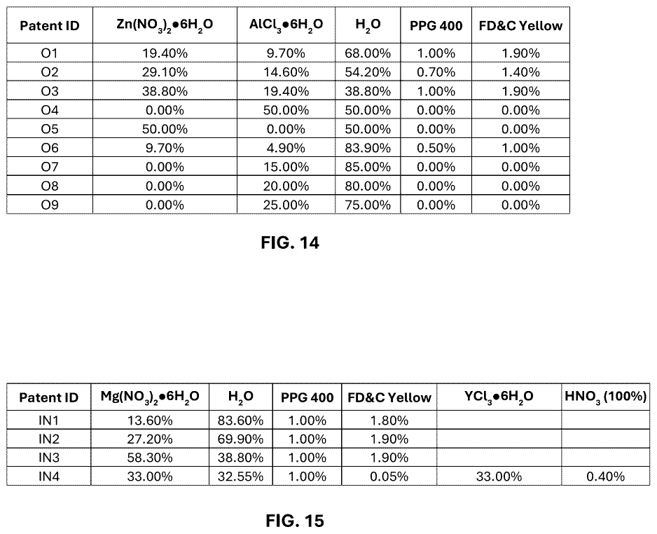

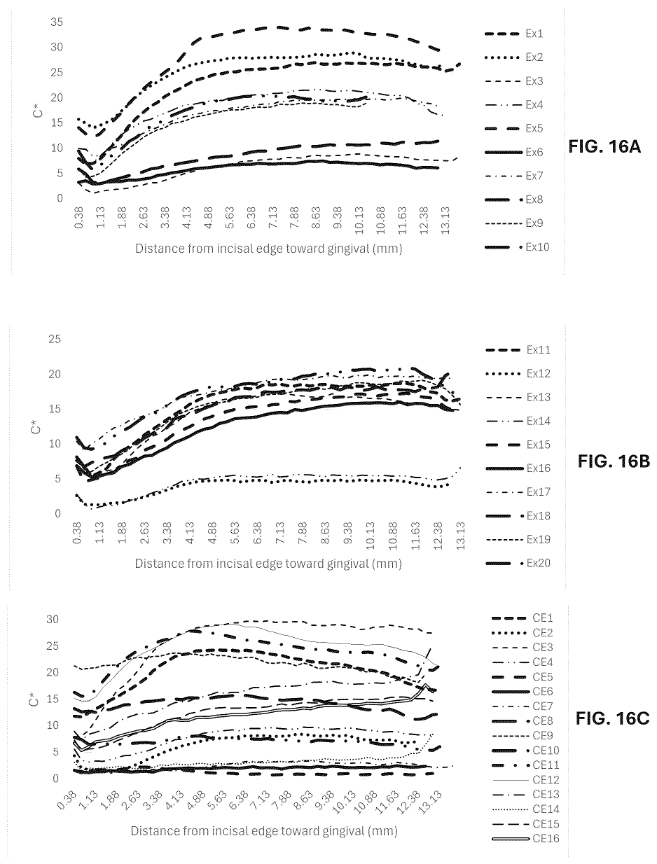

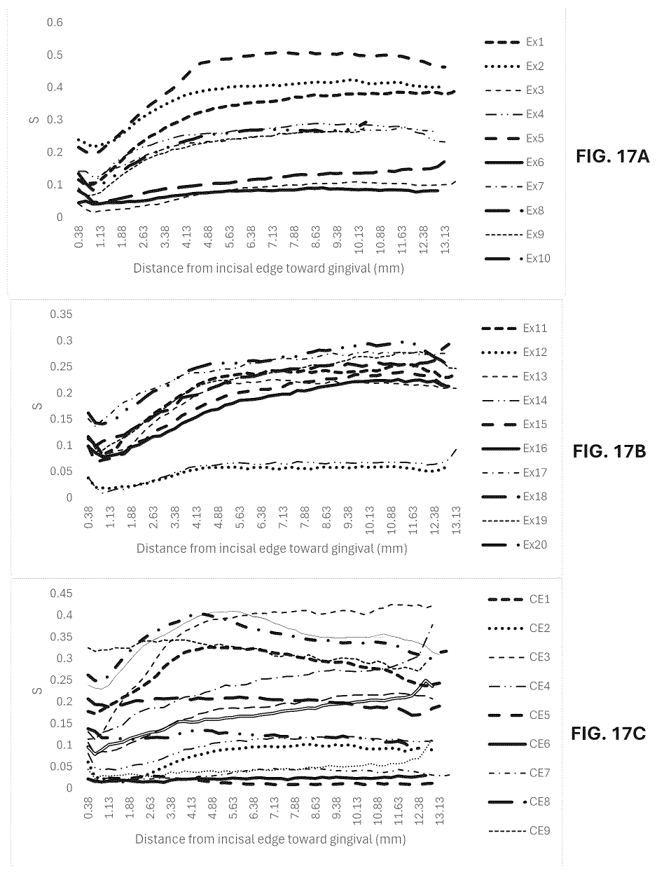

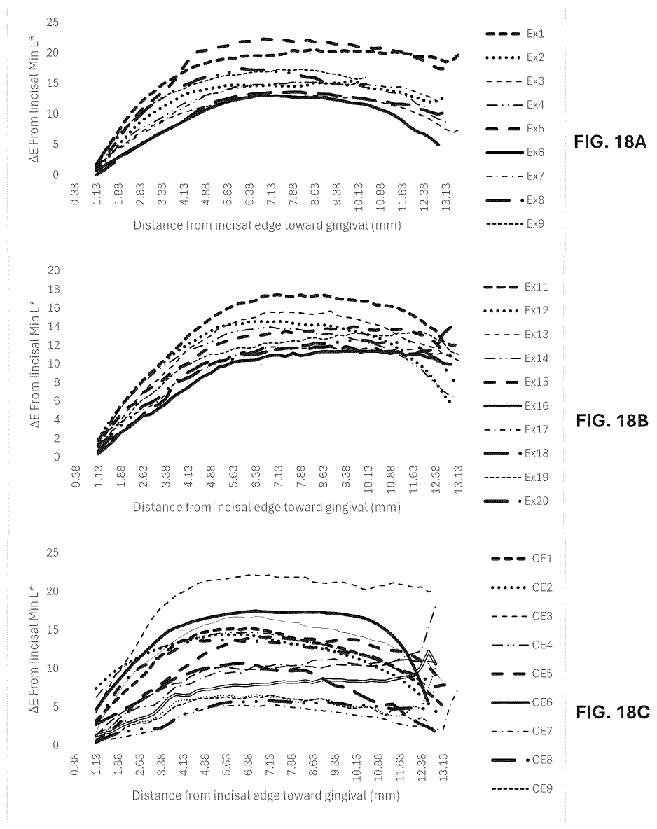

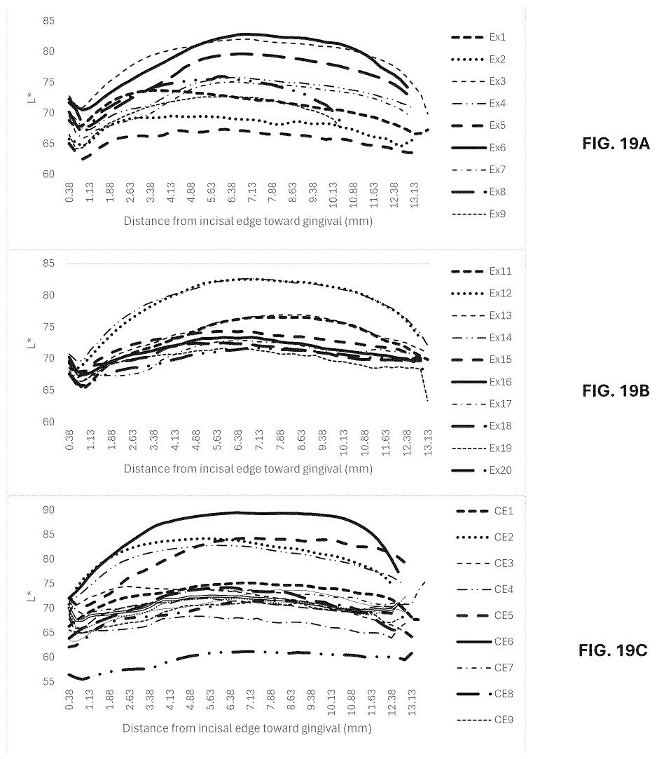

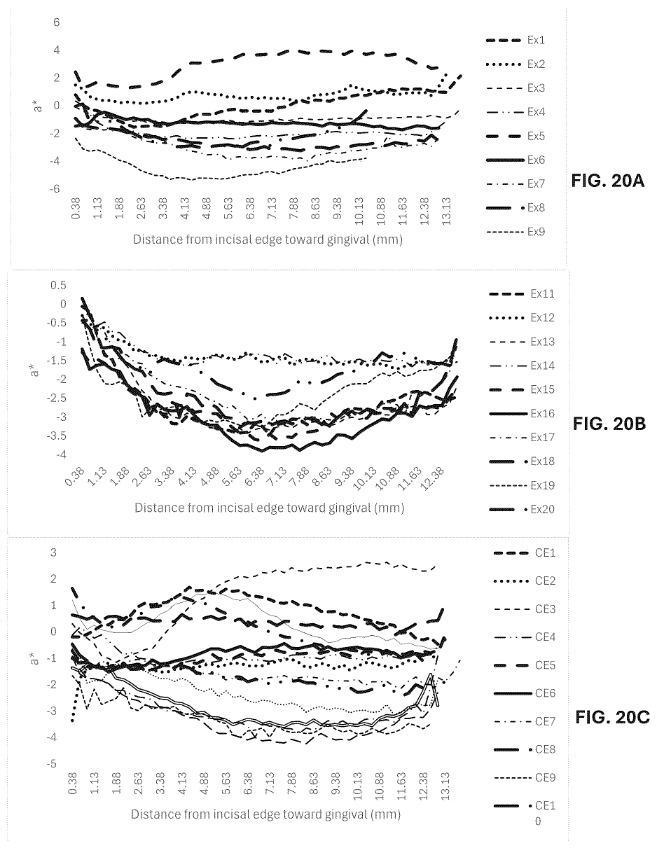

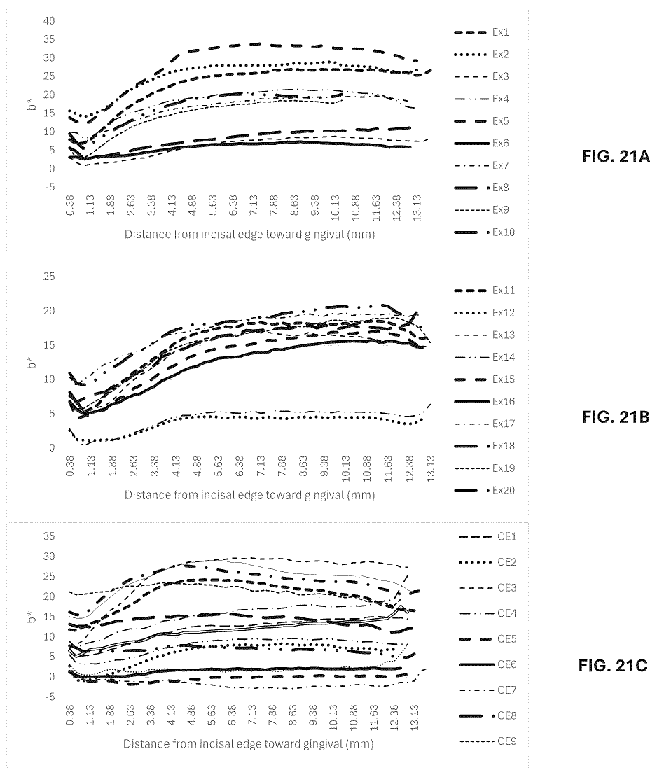

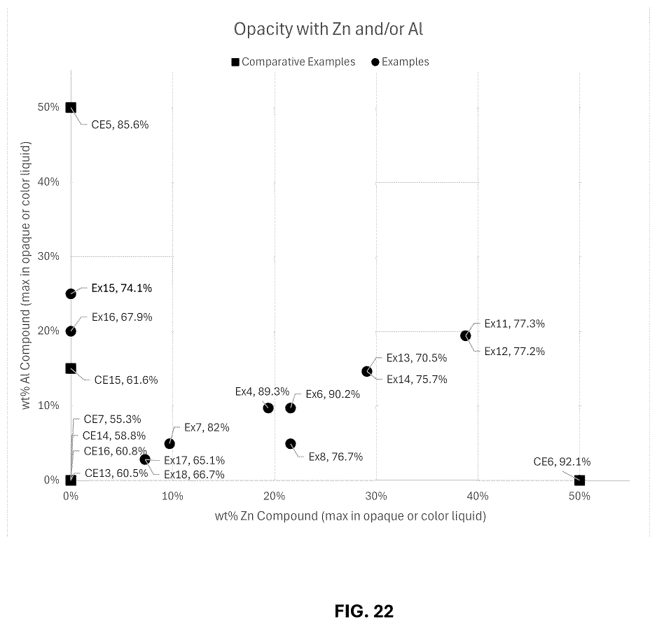

Inventive and comparative examples of composition application methods are listed in FIGS. 11A-11B and 13-15. “#Y” is the mole ratio of yttria in the yttria-stabilized zirconia dental ceramic.

FIG. 1. An inventive example should satisfy all five of the aspects as defined in FIG. 1. Missing any one or more of the aspects is identified as a counter example (i.e., comparative example), which does not qualify under the natural looking crown definition.

FIG. 3. The slope of L* value and Chroma value are calculated based on the calculation as follows:

$$\dot{{L}^{*}} = \frac{( {L}_{1}^{*} - {L}_{0}^{*} )}{( {l}_{1} - {l}_{0} )}$$

$$\dot{{C}^{*}} = \frac{( \sqrt{{a}_{1}^{* 2} + {b}_{1}^{* 2}} - \sqrt{{a}_{0}^{* 2} + {b}_{0}^{* 2}} )}{( {l}_{1} - {l}_{0} )}$$

L₀* is the L* value from the first measurement point
L₁* is the lowest L* value measured within 1 mm of the incisal edge
a*₀, b*₀ are the corresponding a* value and b* value at L₀*
a*₁, b*₁ are the corresponding a* value and b* value at L₁*
l₀ is the distance from incisal at the first measurement point
l₁ is the distance from incisal at the lowest L* value measured within 1 mm of the incisal edge.

The very first measurement point and the point where L* value has the lowest value within 1 mm of the incisal edge were selected. The difference in L* value divided by the distance in between is the value of \dot{L}*. The a* and b* value at the same two points were selected and calculated to get Ċ*. A sample with negative value of \dot{L}* and Ċ* qualifies for the aspect 1 of natural appearance.

FIG. 4. The a* and b* at the point with lowest L* value near the 33% of the restoration length are selected and the average L*, a*, b* value were taken from middle 45% to 55% of the restoration length. AE between these two value is calculated as follow:

$$Δ E = {( Δ {L}^{* 2} + Δ {a}^{* 2} + Δ {b}^{* 2} )}^{1 / 2}$$

wherein,
ΔL*—lightness difference
Δb*—yellowness or blueness difference, and
Δa*—redness or greenness difference
   A ΔE value of greater than 7 between the point of lowest lightness in the 33% of restoration length region and the middle 45-55% of the restorations satisfy the aspect 2 of natural appearance.

FIG. 5. The C* at the point with lowest L* value near the 33% of the restoration length are selected and the average L*and C* values were taken between middle 45% and 55% of the restoration length. Then calculate the Saturation value for both value as follows:

$$S = \frac{{C}^{*}}{{L}^{*}}$$

Afterwards, use the equation below to calculate the ratio between the saturation at incisal with minimum L* value and the average saturation between middle 45% and 55% of the restoration length:

$$R = \frac{{S}_{incisal min}}{{S}_{middle}} \times 100 \%$$

To satisfy the aspect 3 of natural appearance, R value should be less than 70%.

FIG. 6. The C* and S values are calculated at 33% of the restoration length. The average of C* and S value is calculated from 33% to 67% of the restorations use formulation mentioned previously. An example should qualify the condition that C* and S value at 33% of the restoration length is less than the average C* and S value between 33% and 67% of the restoration length.

FIG. 7. The Opacity values are calculated from the minimum Y luminance factors for spectrophotometric scans taken across the middle 40-80% of the prosthesis length relative to the incisal edge. The ratio of the minimum Y luminance factor taken for a scan across the prosthesis with a black stump to that of a scan across the empty prosthesis yields a percentage opacity. An example should qualify the condition that the opacity be no less than 650% when evaluated in this manner.

In view of the many possible embodiments to which the principles of the disclosed subject matter may be applied, it should be recognized that the illustrated embodiments are only preferred examples of the invention and should not be taken as limiting the scope of the invention.

What is claimed is:

1. A method for coloring a ceramic dental prosthesis, wherein the ceramic dental prosthesis has a total length, comprising:
applying at least one coating of a liquid colorant composition to a portion of an external surface area of the ceramic dental prosthesis, wherein the liquid colorant composition comprises at least one opaquing agent and/or at least one coloring agent;
applying at least one coating of an opaque composition to at least a portion of an internal surface of the ceramic dental prosthesis, wherein the opaque composition comprises at least one opaquing agent; and
sintering the coated ceramic dental prosthesis to obtain a fully sintered ceramic body having a density that is at least 98% of the theoretical density,
wherein the coloring of the ceramic dental prosthesis provides:
a drop in L* and C* as defined by a negative slope of L* and C* vs distance from incisal edge to within 1 mm of the incisal edge;
a point of lowest lightness in the 33% of the total length of the ceramic dental prosthesis that is distinguishable with a ΔE of greater than 7 due to both a lower L* and C* when compared with a middle 45-55% of the ceramic dental prosthesis length;
a saturation at a translucent opal halo of the incisal (minimum L*) of no more than 70% that of the middle 45-55% of the ceramic dental prosthesis length;
an average C* and S throughout the middle third of the ceramic dental prosthesis length that is greater than or equal to C* and S at the border of middle 45-55% of the total length of the ceramic dental prosthesis and 33% of the total length of the ceramic dental prosthesis near the incisal region; and
opacity of no less than 65% in the middle 40-80% of the total length of the ceramic dental prosthesis relative to the incisal edge.

2. The method of claim 1, wherein the liquid colorant composition comprises Zn having a concentration of 250 mmol/L to 600 mmol/L and of Al having a concentration of 100 mmol/L to 300 mmol/L and also comprises Fe.

3. The method of claim 1, wherein the liquid colorant composition comprises Zn having a concentration of 400 mmol/L to 1200 mmol/L and also comprises Fe.

4. The method of claim 1, wherein the opaque composition comprises Al in a concentration of 600 mmol/L to 2000 mmol/L.

5. The method of claim 1, wherein either the liquid colorant composition or the opaque composition comprises Zn having a concentration of 400 mmol/L to 2800 mmol/L and Al having a concentration of 250 mmol/L to 2000 mmol/L and resulting in an opacity of no less than 70% in the middle 40-80% of the length of the sintered dental ceramic prosthesis relative to the incisal edge.

6. The method of claim 1, further comprising, prior to applying at least one coating of a liquid colorant composition, applying at least one coating of a liquid incisal composition to an external incisal surface area of the ceramic dental prosthesis, the liquid incisal composition comprising a Mg component.

7. The method of claim 6, comprising applying a first coating of the liquid colorant composition from a gingival edge to an edge line of the previously coated incisal surface area; applying a second coating of the liquid colorant composition from the gingival edge to an upper line that is lower than the edge line of the first layer of colorant composition; and applying a third layer of the liquid colorant composition to coat 40-70% of the surface, starting from the gingival edge.

8. The method of claim 7, comprising applying the coatings in a pattern to provide a lobes effect.

9. The method of claim 1 wherein more than one coating of the opaque composition is applied.

10. The method of claim 6, wherein the incisal composition comprises 5 wt % to 70 wt % Mg(NO₃)₂·(H₂O)₆, based on the total weight of the incisal composition.

11. The method of claim 1, wherein the colorant composition comprises 0.3 wt % to 2.5 wt % of an Fe-containing coloring agent, and 0.3 wt % to 2 wt % a Ni-containing coloring agent, based on the total weight of the composition.

12. The method of claim 1, wherein the colorant composition comprises:
(a) 5 wt % to 10 wt % of the first opaquing agent comprising Zn, based on the total weight of the composition;
(b) 0.5 wt % to 5 wt % of the second opaquing agent comprising Al, based on the total weight of the composition;
(c) 3 wt % to 15 wt % of a first coloring agent comprising Fe, based on the total weight of the composition;
(d) 0.1 wt % to 2 wt % of a second coloring agent comprising Ni, based on the total weight of the composition;
(e) at least one wetting agent; and
(f) at least one solvent.

13. The method of claim 1, wherein the liquid opaque composition comprises aluminum chloride and zinc nitrate.

14. The method of claim 13, wherein the liquid opaque composition comprises 0.4 wt % to 40 wt % of AlCl₃.6H₂O and 5 wt % to 40 wt % of Zn(NO₃)₂·6H₂O, based on the total weight of the liquid opaque composition.

15. The method of claim 1, wherein the ceramic dental prosthesis has a transmittance at the incisal greater than 60% at 700 nm when measured on a 1 mm thick sintered body.

16. The method of claim 1, wherein the ceramic dental prosthesis is a yttria-stabilized zirconia dental ceramic having greater than 5 mol % yttria.

17. A coloring system kit for coloring a ceramic dental prosthesis comprising:
(I) at least 4 unique liquid colorant compositions, wherein each colorant composition comprises:
(a) a Zn(NO₃)₂·6H₂O opaquing agent;
(b) an AlCl₃·6H₂O opaquing agent;
(c) an Fe(NO₃)₃·9H₂O coloring agent;
(d) a Ni(NO₃)₂·6H₂O coloring agent;
(e) polypropylene glycol; and
(g) at least one solvent;
(II) at least one unique liquid opaque composition, wherein each opaque composition comprises at least one opaquing agent; and
(III) a liquid incisal solution comprising a Mg component.

18. A method comprising:
testing a ceramic dental prosthesis, wherein the ceramic dental prosthesis has a total length, for color criteria:
a drop in L* and C* as defined by a negative slope of L* and C* vs distance from incisal edge to within 1 mm of the incisal edge;
a point of lowest lightness in the 33% of the total length of the ceramic dental prosthesis that is distinguishable with a ΔE of greater than 7 due to both a lower L* and C* when compared with a middle 45-55% of the ceramic dental prosthesis length;
a saturation at a translucent opal halo of the incisal (minimum L*) of no more than 70% that of the middle 45-55% of the ceramic dental prosthesis length; and
an average C* and S throughout the middle third of the ceramic dental prosthesis length that is greater than or equal to C* and S at the border of middle 45-55% of the total length of the ceramic dental prosthesis and 33% of the total length of the ceramic dental prosthesis near the incisal region;
opacity of no less than 65% in the middle 40-80% of the total length of the ceramic dental prosthesis relative to the incisal edge.
and providing a ceramic dental prosthesis that satisfies the color criteria to an end-user.

19. A sintered body shaped as an anterior tooth and comprising 5 to 8 mol % yttria stabilized zirconia and Al, Zn, and Fe as coloring pigments, comprising:
a drop in L* and C* as defined by a negative slope of L* and C* vs distance from incisal edge to within 1 mm of an incisal edge;
a point of lowest lightness in the 33% of a total length of the ceramic dental prosthesis that is distinguishable with a ΔE of greater than 7 due to both a lower L* and C* when compared with a middle 45-55% of the ceramic dental prosthesis length;
a saturation at a translucent opal halo of the incisal (minimum L*) of no more than 70% that of the middle 45-55% of the ceramic dental prosthesis length; and
an average C* and S throughout the middle third of the ceramic dental prosthesis length that is greater than or equal to C* and S at the border of middle 45-55% of the total length of the ceramic dental prosthesis and 33% of the total length of the ceramic dental prosthesis near the incisal region;
opacity of no less than 65% in the middle 40-80% of the total length of the ceramic dental prosthesis relative to the incisal edge.
